SEQUENCE LISTING (89 sequences)

SEQ ID NO: 1 - protein, 8 aa, Artificial Sequence
  MT1-MMP cleavage site employed in the recombinant proteins
VPLSLRSG

SEQ ID NO: 2 - protein, 637 aa, Artificial Sequence
  TAT-TLK1B fusion protein
MHHHHHHSSGLVPRGSGMKETAAAKFERQHMDSPDLGTDDDDKAMAISDPNSSSVDKLGYGRKKRRQRRRGGSTMSGYPYDVPDYAGSMVKLAALESNKIQDLEKKEGRIDDLLRANCDLRRQIDEQQKLLEKYKERLNKCISMSKKLLIEKSTQEKLSSREKSMQDRLRLGHFTTVRHGASFTEQWTDGFAFQNLVKQQEWVNQQREDIERQRKLLAKRKPPTANNSQAPSTNSEPKQRKNKAVNGAENDPFVRPNLPQLLTLAEYHEQEEIFKLRLGHLKKEEAEIQAELERLERVRNLHIRELKRINNEDNSQFKDHPTLNERYLLLHLLGRGGFSEVYKAFDLYEQRYAAVKIHQLNKSWRDEKKENYHKHACREYRIHKELDHPRIVKLYDYFSLDTDTFCTVLEYCEGNDLDFYLKQHKLMSEKEARSIVMQIVNALRYLNEIKPPIIHYDLKPGNILLVDGTACGEIKITDFGLSKIMDDDSYGVDGMDLTSQGAGTYWYLPPECFVVGKEPPKISNKVDVWSVGVIFFQCLYGRKPFGHNQSQQDILQENTILKATEVQFPVKPVVSSEAKAFIRRCLAYRKEDRFDVHQLANDPYLLPHMRRSNSSGNLHMAGLTASPTPPSSSIITY

SEQ ID NO: 3 - protein, 648 aa, Artificial Sequence
  TAT-TLK-MS fusion protein
MHHHHHHSSGLVPRGSGMKETAAAKFERQHMDSPDLGTDDDDKAMAISDPNSSSVDKLGYGRKKRRQRRRGGSTMSGYPYDVPDYAGSMVKLAALESNKIQDLEKKEGRIDDLLRANCDLRRQIDEQQKLLEKYKERLNKCISMSKKLLIEKSTQEKLSSREKSMQDRLRLGHFTTVRHGASFTEQWTDGFAFQNLVKQQEWVNQQREDIERQRKLLAKRKPPTANNSQAPSTNSEPKQRKNKAVNGAENDPFVRPNLPQLLTLAEYHEQEEIFKLRLGHLKKEEAEIQAELERLERVRNLHIRELKRINNEDNSQFKDHPTLNERYLLLHLLGRGGFSEVYKAFDLYEQRYAAVKIHQLNKSWRDEKKENYHKHACREYRIHKELDHPRIVKLYDYFSLDTDTFCTVLEYCEGNDLDFYLKQHKLMSEKEARSIVMQIVNALRYLNEIKPPIIHYDLKPGNILLVDGTACGEIKITDFGLSKIMDDDSYGVDGMDLTSQGAGTYWYLPPECFVVGKEPPKISNKVDVWSVGVIFFQCLYGRKPFGHNQSQQDILQENTILKATEVQFPVKGSVPLSLRSGTPVVSSEAKAFIRRCLAYRKEDRFDVHQLANDPYLLPHMRRSNSSGNLHMAGLTASPTPPSSSIITY

SEQ ID NO: 4 - protein, 650 aa, Artificial Sequence
  TAT-TL-MS-K fusion protein
MHHHHHHSSGLVPRGSGMKETAAAKFERQHMDSPDLGTDDDDKAMAISDPNSSSVDKLGYGRKKRRQRRRGGSTMGGYPYDVPDYAGSMLKLAALESNKIQDLEKKEGRIDDLLRANCDLRRQIDEQQKLLEKYKERLNKCISMSKKLLIEKSTQEKLSSREKSMQDRLRLGHFTTVRHGASFTEQWTDGFAFQNLVKQQEWVNQQREDIERQRKLLAKRKPPTANNSQAPSTNSEPKQRKNKAVNGAENDPFVRPNLPQLLTLAEYHEQEEIFKLRLGHLKKEEAEIQAELERLERVRNLHIRELKRINNEDNSQFKAAAVPLSLRSGTSDHPTLNERYLLLHLLGRGGFSEVYKAFDLYEQRYAAVKIHQLNKSWRDEKKENYHKHACREYRIHKELDHPRIVKLYDYFSLDTDTFCTVLEYCEGNDLDFYLKQHKLMSEKEARSIVMQIVNALRYLNEIKPPIIHYDLKPGNILLVDGTACGEIKITDFGLSKIMDDDSYGVDGMDLTSQGAGTYWYLPPECFVVGKEPPKISNKVDVWSVGVIFFQCLYGRKPFGHNQSQQDILQENTILKATEVQFPVKPVVSSEAKAFIRRCLAYRKEDRFDVHQLANDPYLLPHMRRSNSSGNLHMAGLTASPTPPSSSIITY

SEQ ID NO: 5 - protein, 5 aa, Artificial Sequence
  Furin cleavage recognition sequence
RRKRY

SEQ ID NO: 6 - protein, 8 aa, Artificial Sequence
  Alternative MMP cleavage site
VPLSLTMG

SEQ ID NO: 7 - protein, 8 aa, Artificial Sequence
  Alternative MMP cleavage site
GPLSLTMG

SEQ ID NO: 8 - protein, 8 aa, Artificial Sequence
  Alternative MMP cleavage site
GVLSLTMG

SEQ ID NO: 9 - protein, 8 aa, Artificial Sequence
  Alternative MMP cleavage site
GGLSLTMG

SEQ ID NO: 10 - protein, 8 aa, Artificial Sequence
  Alternative MMP cleavage site
VHLSLTMG

SEQ ID NO: 11 - protein, 8 aa, Artificial Sequence
  Alternative MMP cleavage site
VPQSLTMG

SEQ ID NO: 12 - protein, 8 aa, Artificial Sequence
  Alternative MMP cleavage site
VPRSLTMG

SEQ ID NO: 13 - protein, 8 aa, Artificial Sequence
  Alternative MMP cleavage site
VPLGLTMG

SEQ ID NO: 14 - protein, 8 aa, Artificial Sequence
  Alternative MMP cleavage site
VPLSITMG

SEQ ID NO: 15 - protein, 8 aa, Artificial Sequence
  Alternative MMP cleavage site
VPLSLAMG

SEQ ID NO: 16 - protein, 8 aa, Artificial Sequence
  Alternative MMP cleavage site
VPLSLDMG

SEQ ID NO: 17 - protein, 8 aa, Artificial Sequence
  Alternative MMP cleavage site
VPLSLTGG

SEQ ID NO: 18 - protein, 8 aa, Artificial Sequence
  Alternative MMP cleavage site
GPQGIAGQ

SEQ ID NO: 19 - protein, 8 aa, Artificial Sequence
  Alternative MMP cleavage site
GPQGIAGQ

SEQ ID NO: 20 - protein, 8 aa, Artificial Sequence
  Alternative MMP cleavage site
VPMSMRGG

SEQ ID NO: 21 - protein, 8 aa, Artificial Sequence
  Alternative MMP cleavage site
IPVSLRSG

SEQ ID NO: 22 - protein, 8 aa, Artificial Sequence
  Alternative MMP cleavage site
RPFSMIMG

SEQ ID NO: 23 - protein, 8 aa, Artificial Sequence
  Alternative MMP cleavage site
VPLSLTMG

SEQ ID NO: 24 - protein, 8 aa, Artificial Sequence
  Alternative MMP cleavage site
VPLSLYSG

SEQ ID NO: 25 - protein, 8 aa, Artificial Sequence
  Alternative MMP cleavage site
IPESLRAG

SEQ ID NO: 26 - protein, 8 aa, Artificial Sequence
  Alternative MMP cleavage site
IPENFFGV

SEQ ID NO: 27 - protein, 8 aa, Artificial Sequence
  Alternative MMP cleavage site
VPYGLGSP

SEQ ID NO: 28 - protein, 8 aa, Artificial Sequence
  Alternative MMP cleavage site
HPSAFSEA

SEQ ID NO: 29 - protein, 8 aa, Artificial Sequence
  Alternative MMP cleavage site
GPQGIAGQ

SEQ ID NO: 30 - protein, 8 aa, Artificial Sequence
  Alternative MMP cleavage site
GPQGLLGA

SEQ ID NO: 31 - protein, 8 aa, Artificial Sequence
  Alternative MMP cleavage site
GPAGLSVL

SEQ ID NO: 32 - protein, 8 aa, Artificial Sequence
  Alternative MMP cleavage site
GPAGIVTK

SEQ ID NO: 33 - protein, 8 aa, Artificial Sequence
  Alternative MMP cleavage site
DAASLLGL

SEQ ID NO: 34 - protein, 8 aa, Artificial Sequence
  Alternative MMP cleavage site
RPAVMTSP

SEQ ID NO: 35 - protein, 8 aa, Artificial Sequence
  Alternative MMP cleavage site
PPGAYHGA

SEQ ID NO: 36 - protein, 8 aa, Artificial Sequence
  Alternative MMP cleavage site
LRAYLLPA

SEQ ID NO: 37 - protein, 8 aa, Artificial Sequence
  Alternative MMP cleavage site
GPYELKAL

SEQ ID NO: 38 - protein, 8 aa, Artificial Sequence
  Alternative MMP cleavage site
TAAALTSC

SEQ ID NO: 39 - protein, 8 aa, Artificial Sequence
  Alternative MMP cleavage site
GPEGLRVG

SEQ ID NO: 40 - protein, 8 aa, Artificial Sequence
  Alternative MMP cleavage site
GHARLVHV

SEQ ID NO: 41 - protein, 8 aa, Artificial Sequence
  Alternative MMP cleavage site
QPVGINTS

SEQ ID NO: 42 - protein, 8 aa, Artificial Sequence
  Alternative MMP cleavage site
ELGTYNVI

SEQ ID NO: 43 - protein, 8 aa, Artificial Sequence
  Alternative MMP cleavage site
DVAQFVLY

SEQ ID NO: 44 - protein, 8 aa, Artificial Sequence
  Alternative MMP cleavage site
DVANYNFF

SEQ ID NO: 45 - protein, 8 aa, Artificial Sequence
  Alternative MMP cleavage site
HPVGLLAR

SEQ ID NO: 46 - protein, 8 aa, Artificial Sequence
  Alternative MMP cleavage site
KPQQFFGL

SEQ ID NO: 47 - protein, 8 aa, Artificial Sequence
  Alternative MMP cleavage site
IPVSLRSG

SEQ ID NO: 48 - protein, 8 aa, Artificial Sequence
  Alternative MMP cleavage site
HPVGLLAR

SEQ ID NO: 49 - protein, 8 aa, Artificial Sequence
  Alternative MMP cleavage site
QPVGINTS

SEQ ID NO: 50 - protein, 8 aa, Artificial Sequence
  Alternative MMP cleavage site
RPAVMTSP

SEQ ID NO: 51 - protein, 8 aa, Artificial Sequence
  Alternative MMP cleavage site
GPQGIAGQ

SEQ ID NO: 52 - protein, 8 aa, Artificial Sequence
  Alternative MMP cleavage site
HVLNLRST

SEQ ID NO: 53 - protein, 8 aa, Artificial Sequence
  Alternative MMP cleavage site
DPESIRSE

SEQ ID NO: 54 - protein, 8 aa, Artificial Sequence
  Alternative MMP cleavage site
DPLEFKSH

SEQ ID NO: 55 - protein, 8 aa, Artificial Sequence
  Alternative MMP cleavage site
RPIPITAS

SEQ ID NO: 56 - protein, 8 aa, Artificial Sequence
  Alternative MMP cleavage site
RVLGLKAH

SEQ ID NO: 57 - protein, 8 aa, Artificial Sequence
  Alternative MMP cleavage site
KVLNLTDN

SEQ ID NO: 58 - protein, 8 aa, Artificial Sequence
  Alternative MMP cleavage site
PPEALRGI

SEQ ID NO: 59 - protein, 8 aa, Artificial Sequence
  Alternative MMP cleavage site
IVAMLRAP

SEQ ID NO: 60 - protein, 8 aa, Artificial Sequence
  Alternative MMP cleavage site
TAAAITGA

SEQ ID NO: 61 - protein, 10 aa, Artificial Sequence
  Alternative MMP cleavage site
SGRIGFLRTA

SEQ ID NO: 62 - protein, 10 aa, Artificial Sequence
  Alternative MMP cleavage site
SGAIGFLRTA

SEQ ID NO: 63 - protein, 10 aa, Artificial Sequence
  Alternative MMP cleavage site
SGRAMHMYTA

SEQ ID NO: 64 - protein, 10 aa, Artificial Sequence
  Alternative MMP cleavage site
SGAAMHMYTA

SEQ ID NO: 65 - protein, 10 aa, Artificial Sequence
  Alternative MMP cleavage site
SGRSENIRTA

SEQ ID NO: 66 - protein, 10 aa, Artificial Sequence
  Alternative MMP cleavage site
SGASENIRTA

SEQ ID NO: 67 - protein, 10 aa, Artificial Sequence
  Alternative MMP cleavage site
SGRPENIRTA

SEQ ID NO: 68 - protein, 10 aa, Artificial Sequence
  Alternative MMP cleavage site
SGAPENIRTA

SEQ ID NO: 69 - protein, 10 aa, Artificial Sequence
  Alternative MMP cleavage site
SGARYRWLTA

SEQ ID NO: 70 - protein, 10 aa, Artificial Sequence
  Alternative MMP cleavage site
SGLISHSITA

SEQ ID NO: 71 - protein, 9 aa, Artificial Sequence
  Alternative MMP cleavage site
SGNLRSKLT

SEQ ID NO: 72 - protein, 10 aa, Artificial Sequence
  Alternative MMP cleavage site
SGVFSIPLTA

SEQ ID NO: 73 - protein, 10 aa, Artificial Sequence
  Alternative MMP cleavage site
SGIKYHSLTA

SEQ ID NO: 74 - protein, 13 aa, Artificial Sequence
  Cell-penetrating peptide
GRKKRRQRRRPPQ

SEQ ID NO: 75 - protein, 7 aa, Artificial Sequence
  Cell-penetrating peptide
RKKRRQR

SEQ ID NO: 76 - protein, 9 aa, Artificial Sequence
  Cell-penetrating peptide
AKKRRQRRR

SEQ ID NO: 77 - protein, 8 aa, Artificial Sequence
  Cell-penetrating peptide
RRRRRRRR

SEQ ID NO: 78 - protein, 27 aa, Artificial Sequence
  Cell-penetrating peptide
GWTLNSAGYLLGKINLKALAALAKKIL

SEQ ID NO: 79 - protein, 18 aa, Artificial Sequence
  Cell-penetrating peptide
LLGKINLKALAALAKKIL

SEQ ID NO: 80 - protein, 13 aa, Human immunodeficiency virus
  TAT peptide residues 47-57
GYGRKKRRQRRRG

SEQ ID NO: 81 - protein, 4 aa, Homo sapiens
  Pro-MT1-MMP
RRKR

SEQ ID NO: 82 - protein, 4 aa, Homo sapiens
  Serum Albumin
VFRR

SEQ ID NO: 83 - protein, 4 aa, Homo sapiens
  C-type natriuretic peptide
RLLR

SEQ ID NO: 84 - protein, 6 aa, Homo sapiens
  Integrin alpha 4 precursor furin cleavage site
HVISKR

SEQ ID NO: 85 - protein, 6 aa, Homo sapiens
  Pro-parathyroid hormone
KSVKKR

SEQ ID NO: 86 - protein, 6 aa, Homo sapiens
  Vascular endothelial growth factor c precursor
HSIIRR

SEQ ID NO: 87 - protein, 6 aa, Homo sapiens
  Albumin precursor
RGVFRR

SEQ ID NO: 88 - protein, 6 aa, Homo sapiens
  Furin self-cleavage site
RGVTKR

SEQ ID NO: 89 - protein, 641 aa, Artificial Sequence
  TAT-MS-TLK1B
MHHHHHHSSGLVPRGSGMKETAAAKFERQHMDSPDLGTDDDDKAMAISDPNSSSVDKLGYGRKKRRQRRRGGSVPLSLRSGTYPYDVPDYASMVKLAALESNKIQDLEKKEGRIDDLLRANCDLRRQIDEQQKLLEKYKERLNKCISMSKKLLIEKSTQEKLSSREKSMQDRLRLGHFTTVRHGASFTEQWTDGFAFQNLVKQQEWVNQQREDIERQRKLLAKRKPPTANNSQAPSTNSEPKQRKNKAVNGAENDPFVRPNLPQLLTLAEYHEQEEIFKLRLGHLKKEEAEIQAELERLERVRNLHIRELKRINNEDNSQFKDHPTLNERYLLLHLLGRGGFSEVYKAFDLYEQRYAAVKIHQLNKSWRDEKKENYHKHACREYRIHKELDHPRIVKLYDYFSLDTDTFCTVLEYCEGNDLDFYLKQHKLMSEKEARSIVMQIVNALRYLNEIKPPIIHYDLKPGNILLVDGTACGEIKITDFGLSKIMDDDSYGVDGMDLTSQGAGTYWYLPPECFVVGKEPPKISNKVDVWSVGVIFFQCLYGRKPFGHNQSQQDILQENTILKATEVQFPVKPVVSSEAKAFIRRCLAYRKEDRFDVHQLANDPYLLPHMRRSNSSGNLHMAGLTASPTPPSSSIITY

(19) United States
(12) Patent Application Publication
(10) Pub. No.: US 2016/0067316 A1
(43) Pub. Date: Mar. 10, 2016

(54) Selective Sensitization of Cancer Cells to, and Selective Protection of Non-Cancer Cells from Genotoxic Therapies

(71) Applicant: Gulshan N. Sunavala-Dossabhoy, Shreveport, LA (US)

(72) Inventor: Gulshan N. Sunavala-Dossabhoy, Shreveport, LA (US)

(73) Assignee: Board of Supervisors of Louisiana State University and Agricultural and Mechanical College, Shreveport, LA (US)

(21) Appl. No.: 14/849,131

(22) Filed: Sep. 9, 2015

Related U.S. Application Data
(60) Provisional application No. 62/048,314, filed on Sep. 10, 2014.

(51) Int. Cl. A61K 38/45; C12N 7/00; C12N 9/12

(52) U.S. Cl. CPC A61K 38/45; C12N 9/12; C12N 7/00; C12Y 207/11001; C12N 2740/16322

(57) ABSTRACT

Compositions and methods are disclosed that employ Tousled-like kinase to selectively protect normal tissues from the adverse effects of radiation therapy, genotoxic chemotherapy and the like without protecting cancer tissues; or to sensitize cancer cells to such cancer therapies without similarly sensitizing normal tissues; or both protect normal cells and sensitize cancer cells. The compositions and methods may be used as a complementary therapy to selectively reduce adverse effects of the principal therapy (radiation therapy or chemotherapy) in normal tissues, with minimal impact on the antitumor effects of the principal therapy.

The benefit of the filing date of U.S. provisional application 62/048,314, filed Sep. 10, 2014, is claimed under 35 U.S.C. §119(e). The complete disclosure of the priority application is hereby incorporated by reference.

This invention was made with Government support under grant numbers R03 CA169959 and R21 CA173162 awarded by the National Cancer Institute, National Institutes of Health. The Government has certain rights in the invention.

The invention pertains to compositions and methods for selectively sensitizing cancer cells, for example head and neck squamous cell carcinomas, with radio- and chemo-therapeutic agents, while sparing normal cells from the effect of those agents. The invention also pertains to complementary therapies for cancer patients, to selectively reduce the adverse effects of radiation therapy or chemotherapy in normal tissues, with minimal impact on the effectiveness of the principal therapy against cancer cells.

Much work in cancer therapy is devoted to the problem of drug resistance. Resistance is a late outcome that could be avoided or reduced with better combination therapies, while reducing radiation or chemotherapy doses and still specifically targeting cancer cells. Cancer therapies such as radiation or chemotherapy often result in collateral destruction or damage to healthy, non-targeted tissues. For example, oral complications associated with cancer therapy afflict almost 100% of patients undergoing radiotherapy for head and neck cancers, about 80% of patients undergoing high-dose full-body radiation before a bone marrow transplant, and about 40% of patients undergoing chemotherapy or radioiodine ablation for thyroid cancer. Common side effects to the head and neck following radiation or chemotherapy include oral mucositis and salivary gland dysfunction. The signs and symptoms include intense pain, oral dryness (xerostomia), dysphagia (difficulty swallowing), ulcerations, soft tissue necrosis, opportunistic infections, and dental caries.

Xerostomia is an agonizing, almost invariable side effect of radiotherapy to the head and neck. It greatly impairs a patient's quality of life. The decline in tissue function continues after radiation, although some recovery can occur after a period of months or years. Symptoms can be so severe as to require parenteral nutrition. Concerns over such collateral effects can compromise what would otherwise be the optimal treatment against the cancer itself.

Previous approaches to ameliorating cancer therapy-induced oral mucositis and salivary gland hypofunction have been directed primarily towards managing symptoms, for example by using emollients, sialogogues, and analgesics. These approaches are of only limited efficacy. Preemptive interventions are often needed to prevent or manage complications. Cytoprotective drugs or biological response modifiers can somewhat reduce the toxicity of chemotherapy or radiation therapy on healthy tissues, but at the risk of protecting tumor tissues as well. A free-radical scavenger, amifostine, has been used concurrently with radiotherapy to avert mucositis and xerostomia. However, poor patient tolerance and concerns about tumor protection have limited its widespread acceptance. Palfermin (recombinant human keratinocyte growth factor) has shown promise in reducing the severity of oral mucositis, but FDA approval only extends to patients undergoing radiotherapy for hematologic malignancies.

There are very few agents in clinical use that are effective in selectively protecting normal tissues during cancer treatments. Toxicity to normal tissues remains a principal limiting factor in curative cancer treatment.

Tousled is an evolutionarily conserved protein found in both plants and animals. Two homologs of Tousled found in humans are cell-cycle dependent kinases: Tousled-like kinase 1, and Tousled-like kinase 2 (TLK1 and TLK2, respectively). The tousled like kinases are involved in chromatin assembly, DNA damage response and repair, and transcription. Two TLK genes exist in humans and their expression is often dysregulated in cancer. TLKs phosphorylate Asf1, histone H3, and Rad9, thereby regulating DNA Damage Response (DDR) and Double-Strand Break (DSB) repair. TLKs maintain genomic stability and are important therapeutic intervention targets. Both TLK1 and TLK2 have maximum activity during DNA replication.

A variant of TLK1, TLK1B, results from an alternative, downstream mRNA translation start site. FIG. 1 depicts a schematic representation of TLK1 mRNA and TLK1B translation from mRNA. As a result of the alternative translation start site 237 amino acids are truncated at the N-terminus of TLK1B as compared to TLK1. (ORF=open reading frame. UTR=untranslated region). Normal cells predominantly express the full-length TLK1. The alternate translation start site that leads to TLK1B is predominantly used in cells that express high levels of eukaryotic translation initiation factor 4E (eIF4E), or phosphorylated eIF4E-binding protein I (eIF4E-BP1). TLK1 and TLK1B play an important role in genotoxin-induced stress response. Their overexpression in immortalized epithelial cells improves survival following ionizing radiation. Further, the exogenous expression of TLK1B does not transform cells or induce tumors in syngeneic mice.

In humans TLK1 regulates chromatin assembly through phosphorylation of the histone chaperone Asf1. The histone H3 and the DNA repair protein Rad9 are additional substrates of the protein, suggesting that TLK1 and TLK1B play a role in DNA remodeling and repair. Recent research suggests that TLK1B functions both as a kinase and as a chaperone. A transient decrease in kinase activity is important for checkpoint establishment following genotoxic damage, and its reestablishment is necessary for exit from cell arrest. The chaperone function plays a role in modulating chromatin at DNA repair sites.

S. Palaniyandi et al., “Adenoviral delivery of Tousled kinase for the protection of salivary glands against ionizing radiation damage,” Gene Ther., vol. 18, pp. 275-282 (2011) reported that adenoviral-mediated human TLK1B expression in rat salivary gland cells protected against the damaging effects of ionizing radiation, both in vitro and in vivo. However, even when the vector was delivered to a specific location in vivo, the vector nevertheless spread and was expressed in other tissues, which could limit its usefulness for clinical purposes.

P. S. Timiri Shanmugam et al., “Recombinant AAV9-TLK1B administration ameliorates fractionated radiation-induced xerostomia,” Hum. Gene Ther., vol. 24, pp. 604-612 (2013) reported that recombinant adeno-associated virus delivery of TLK1B can preserve rat salivary gland function against clinically-relevant fractionated radiation. However, as with any gene therapy approach, there exists a risk of integration into the host genome, or mutation of host genes.

Although “gene therapy” has received much attention, “protein transduction” is an alternate method for efficient delivery of bio-active molecules directly to mammalian cells. “Protein transduction” occurs when a cell internalizes a protein from the external environment into the cell, usually promoted by a cell-penetrating peptide or domain, also referred to as a protein transduction peptide, a protein transduction domain, a membrane translocating peptide, or a membrane translocating domain. Fusion proteins incorporating both the protein of interest and the HIV TAT protein transduction domain (PTD) are sometimes used for this purpose. The HIV TAT-PTD (residues 47-57 of TAT), SEQ ID NO: 80 (GYGRKKRRQRRRG) can efficiently ferry a variety of macromolecules across cell membranes. TAT-PTD can be used to transduce a variety of cell types, both in vitro and in vivo.

Patients with advanced cancers often receive a combination of treatments, which can include both chemotherapy and radiotherapy. For example, cisplatin is often used in conjunction with radiotherapy. Y. Takayama et al., “Silencing of Tousled-like kinase 1 sensitizes cholangiocarcinoma cells to cisplatin-induced apoptosis,” Canc. Lett., vol. 296, pp. 27-34 (2010) reported that the siRNA knockdown of TLK1 in cholangiocarcinoma increased tumor sensitivity to cisplatin-induced apoptosis.

Overexpression of matrix metalloproteinases (MMPs) is characteristic of several types of cancer, including head and neck squamous cell carcinoma (HNSCC). MMPs are a family of zinc-dependent endopeptidases that can collectively degrade all components of the extracellular matrix and the basement membrane. Most MMPs are secreted, but a few are tethered to the cell surface. MT1-MMP, which is sometimes also called MMP-14, is a membrane-type MMP that is highly expressed at the invading or leading edge of cancer cells. MMPs can be overexpressed both in tumor cells and in tumor-associated stromal cells. MT1-MMP is a critical protein that activates MMP-2 and MMP-13, and that also possesses its own collagenolytic activity. MT1-MMP also plays a role in the activation of MMP-9, via the activation of MMP-2. Previous studies have found a high correlation between MMP expression in tumors and metastases. It has been suggested that the proteases that are overexpressed in cancers might be used to activate cytotoxic pro-toxins selectively in the tumor milieu, and thereby to diminish off-target cytotoxicities.

Furin, an intracellular protease that is involved in converting pro-MT1-MMP to active MT1-MMP, is highly expressed in several cancers, including HNSCC. D. Bassi et al., “Elevated furin expression in aggressive human head and neck tumors and tumor cell lines,” Molecular Carcinogenesis, vol. 31, pp. 224-232 (2001) reported a correlation between furin levels and the agressiveness of certain cancers, including head and neck cancers. Observations suggested that furin played a role in MT1-MMP activation in certain cancers.

D. Bassi et al., “Proprotein convertases: ‘Master Switches’ in the regulation of tumor growth and progression,” Molecular Carcinogenesis, vol. 44, pp. 151-161 (2005) reported that furin and another proprotein convertase, PACE4, play a substantial role in tumor growth, invasiveness, and metastasis.

T. Wang et al., “Recombinant immunoproapoptotic proteins with furin site can translocate and kill HER2-positive cancer cells,” Cancer Res., vol. 67, pp. 11830-11839 (2007) described a strategy employing the furin-catalyzed cleavage of an anti-Her2-linked pro-apoptotic protein for the targeted killing of cancer cells.

B. Turk et al., “Determination of protease cleavage site motifs using mixture-based oriented peptide libraries,” Nat. Biotech., vol. 19, pp. 661-667 (2001) discloses several MMP cleavage site motifs and expected MMP cleavage sites. See also S. Kridel et al., “A unique substrate binding mode discriminates membrane type-1 matrix metalloproteinase from other matrix metalloproteinases,” J. Biol. Chem. vol. 277, pp. 23788-23793 (2002).

S. Liu et al., “Intermolecular complementation achieves high specificity tumor targeting by anthrax toxin,” Nat. Biotech., vol. 23, pp. 725-730 (2005) discloses the production of anthrax toxin protective antigen mutants with mutations in different anthrax lethal factor binding subsites, and containing either urokinase plasminogen activator or matrix metalloproteinases cleavage sites. Individually, the mutants had low toxicity due to impaired lethal factor binding, but when administered together to urokinase plasminogen activator- and matrix metalloproteinase-expressing tumor cells, they assembled into functional lethal factor-binding heptamers. The mixture of complementing protective antigen mutants had reduced toxicity to mice and was reported to be effective in the treatment of aggressive transplanted tumors.

Y. Chau et al., “Antitumor efficacy of a novel polymer-peptide drug conjugate in human tumor xenograft models,” Int. J. Cancer, vol. 118, pp. 1519-1526 (2006) discloses a conjugate in which a linker releases the chemotherapeutic drug methotrexate upon cleavage by a matrix metalloproteinase, thereby allowing tumor-targeted delivery.

G. Lee et al., “Peptide-doxorubicin conjugates specifically degraded by matrix metalloproteinases expressed from tumor,” Drug Dev. Res., vol. 67, pp. 438-447 (2006) discloses a conjugate in which a peptide would release the chemotherapeutic drug doxorubicin when the peptide was cleaved by a matrix metalloproteinase to allow targeted anti-cancer drug delivery.

J. Schafer, “Efficient targeting of head and neck squamous cell carcinoma by systemic administration of a dual uPA and MMP-activated engineered anthrax toxin,” PLoS One, vol. 6, e20532 (2011) disclosed the use of an intercomplementing anthrax toxin that required combined cell surface urokinase plasminogen activator and matrix metalloproteinase activities for cellular intoxication, specifically targeting the extracellular signal-regulated kinase/mitogen activated protein kinase pathway for treating head and neck squamous cell carcinoma

G. Sunavala-Dossabhoy et al. “TAT-Mediated delivery of Tousled protein to salivary glands protects against radiation-induced hypofunction,” Int. J. Rad. Onc., vol. 84, pp. 257-265 (2012) describes preclinical animal studies in which cell-permeable TAT-TLK1B, delivered directly to rat salivary glands, was found to mitigate radiation-induced xerostomia. The authors suggested that clinical translation of TAT-TLK1B could be a safer alternative to gene therapy.

WO/2009/088405 discloses a system to protect normal cells against DNA damage using gene therapy to up-regulate the production of TLK1B. Expressed TLK1B protein protects host cells from DNA damage and could be used as a prophylactic to prevent chronic xerostomia resulting from cancer treatment with various genotoxic agents and subsequent salivary gland damage.

WO/2013/119825 (see also pending U.S. application Ser. No. 14/377,457) discloses agents to inhibit the activity of TLK to provide increased sensitivity to radiation and chemotherapeutic agents; and agents to increase the activity of TLK to provide increased protection against DNA damaging agents such as radiation and chemotherapeutic agents.

There is an unfilled need for compositions and methods that allow TLK1B treatment to selectively protect normal tissues from the adverse side effects of radiation therapy and chemotherapy, without protecting cancerous tissues. Higher doses of drugs or ionizing radiation may improve the response rate in some malignancies, but these treatment methods also cause increased toxicity for the patient. There is an unfilled need for improved methods of treating cancer, while minimizing toxic side effects in normal tissues. There is an unfilled need for compositions and methods to supply TLK1B differentially to normal tissue in preference to cancerous tissue.

I have discovered compositions and methods that employ Tousled-like kinase to selectively protect normal tissues from the adverse effects of radiation therapy, genotoxic chemotherapy and the like without protecting cancer tissues; or to sensitize cancer cells to such cancer therapies without similarly sensitizing normal tissues; or both protect normal cells and sensitize cancer cells. The novel approach may be used as a complementary therapy to selectively reduce adverse effects of the principal therapy (radiation therapy or chemotherapy) in normal tissues, with minimal impact on the antitumor effects of the principal therapy.

In prototype embodiments TAT was conjugated to TLK1B to facilitate uptake of TLK1B by cells. Also, an MMP cleavage-sensitive motif was incorporated within the TAT-TLK1B fusion protein. Matrix metalloproteinase (MMP) is typically overexpressed by cancer cells. High concentrations of MMP on cancer cell membranes or in the neighborhood of cancer cells preferentially cleave, and thereby degrade the TAT-TLK1B protein at the membrane of tumor cells or in their vicinity. Whether cleavage occurs in the neighborhood of the cancer cells or at the cell surface, in either case the cleavage differentially inhibits functional TLK1B molecules from accumulating inside cancer cells, while allowing functional TLK1B molecules to be transduced into and retain their function inside normal cells.

The cell-penetrating peptide is preferably TAT, but may alternatively be any of the naturally-occurring or synthetic cell-penetrating peptides known in the art. Curated information concerning known cell-penetrating peptides is readily available and includes, for examples, that found at crdd{dot}osdd{dot}net/raghava/cppsite/index.php and other publicly-accessible websites. As of the filing date of the present application, this curated website contained sequences for 843 cell-penetrating peptides. In other words, a large number of such peptides are known in the art, and any of these cell-penetrating peptides may be used in practicing this invention. Examples of such cell-penetrating peptides, chosen more-or-less arbitrarily for illustrative purposes, include GRKKRRQRRRPPQ (SEQ ID NO: 74), RKKRRQR (SEQ ID NO: 75), AKKRRQRRR (SEQ ID NO: 76), RRRRRRRR (SEQ ID NO: 77), GWTLNSAGYLLGKINLKALAALAKKIL (SEQ ID NO: 78), and LLGKINLKALAALAKKIL (SEQ ID NO: 79). See also the cell-penetrating peptides and motifs disclosed in K. Montrose et al., “Xentry, a new class of cell-penetrating peptide uniquely equipped for delivery of drugs,” Scientific Reports 3:1661 (2013); S. Jones et al., “Characterisation of cell-penetrating peptide-mediated peptide delivery,” Brit. J. Pharm., vol. 145, pp. 1093-1102 (2005); F. Mittelti, “Cell-penetrating peptides: classes, origin, and current landscape,” Drug Disc. Today, vol. 17, pp. 850-860 (2012); and C. Bechara, “Cell-penetrating peptides: 20 years later, where do we stand?” FEBS Letters, vol. 587, pp. 1693-1702 (2013); the complete disclosures of each of which is hereby incorporated by reference.

In lieu of the MT1-MMP cleavage site employed in the prototype embodiments, VPLSLRSG (SEQ ID. NO: 1), the cleavage sequence(s) of other tumor-abundant MMPs may be used, including, for example, those disclosed in B. Turk et al., “Determination of protease cleavage site motifs using mixture-based oriented peptide libraries,” Nat. Biotech., vol. 19, pp. 661-667 (2001); and S. Kridel et al., “A unique substrate binding mode discriminates membrane type-1 matrix metalloproteinase from other matrix metalloproteinases,” J. Biol. Chem. vol. 277, pp. 23788-23793 (2002), the complete disclosures of both of which are hereby incorporated by reference, particularly including but not limited to, the cleavage site motifs disclosed therein. These MMP cleavage sites include, among others: VPLSLTMG (SEQ ID NO: 6), GPLSLTMG (SEQ ID NO: 7), GVLSLTMG (SEQ ID NO: 8), GGLSLTMG (SEQ ID NO: 9), VHLSLTMG (SEQ ID NO: 10), VPQSLTMG (SEQ ID NO: 11), VPRSLTMG (SEQ ID NO: 12), VPLGLTMG (SEQ ID NO: 13), VPLSITMG (SEQ ID NO: 14), VPLSLAMG (SEQ ID NO: 15), VPLSLDMG (SEQ ID NO: 16), VPLSLTGG (SEQ ID NO: 17), GPQGIAGQ (SEQ ID NO: 18), GPQGIAGQ (SEQ ID NO: 19), VPMSMRGG (SEQ ID NO: 20), IPVSLRSG (SEQ ID NO: 21), RPFSMIMG (SEQ ID NO: 22), VPLSLTMG (SEQ ID NO: 23), VPLSLYSG (SEQ ID NO: 24), IPESLRAG (SEQ ID NO: 25), IPENFFGV (SEQ ID NO: 26), VPYGLGSP (SEQ ID NO: 27), HPSAFSEA (SEQ ID NO: 28), GPQGIAGQ (SEQ ID NO: 29), GPQGLLGA (SEQ ID NO: 30), GPAGLSVL (SEQ ID NO: 31), GPAGIVTK (SEQ ID NO: 32), DAASLLGL (SEQ ID NO: 33), RPAVMTSP (SEQ ID NO: 34), PPGAYHGA (SEQ ID NO: 35), LRAYLLPA (SEQ ID NO: 36), GPYELKAL (SEQ ID NO: 37), TAAALTSC (SEQ ID NO: 38), GPEGLRVG (SEQ ID NO: 39), GHARLVHV (SEQ ID NO: 40), QPVGINTS (SEQ ID NO: 41), ELGTYNVI (SEQ ID NO: 42), DVAQFVLY (SEQ ID NO: 43), DVANYNFF (SEQ ID NO: 44), HPVGLLAR (SEQ ID NO: 45), KPQQFFGL (SEQ ID NO: 46), IPVSLRSG (SEQ ID NO: 47), HPVGLLAR (SEQ ID NO: 48), QPVGINTS (SEQ ID NO: 49), RPAVMTSP (SEQ ID NO: 50), GPQGIAGQ (SEQ ID NO: 51), HVLNLRST (SEQ ID NO: 52), DPESIRSE (SEQ ID NO: 53), DPLEFKSH (SEQ ID NO: 54), RPIPITAS (SEQ ID NO: 55), RVLGLKAH (SEQ ID NO: 56), KVLNLTDN (SEQ ID NO: 57), PPEALRGI (SEQ ID NO: 58), IVAMLRAP (SEQ ID NO: 59), TAAAITGA (SEQ ID NO: 60), SGRIGFLRTA (SEQ ID NO: 61), SGAIGFLRTA (SEQ ID NO: 62), SGRAMHMYTA (SEQ ID NO: 63), SGAAMHMYTA (SEQ ID NO: 64), SGRSENIRTA (SEQ ID NO: 65), SGASENIRTA (SEQ ID NO: 66), SGRPENIRTA (SEQ ID NO: 67), SGAPENIRTA (SEQ ID NO: 68), SGARYRWLTA (SEQ ID NO: 69), SGLISHSITA (SEQ ID NO: 70), SGNLRSKLT (SEQ ID NO: 71), SGVFSIPLTA (SEQ ID NO: 72), and SGIKYHSLTA (SEQ ID NO: 73). See also the MMP cleavage sites and motifs disclosed in T. Manon-Jensen, “Mapping of matrix metalloproteinase cleavage sites on syndecan-1 and syndecan-4 ectodomains,” FEBS J., vol. 280, pp. 2320-2331 (2013); and H. Nagase, Substrate specificity of MMPs, Chapter 2, pp. 39-66 in N. Clendeninn et al. (Eds.), Cancer Drug Discovery and Development: Matrix Metalloproteinase Inhibitors in Cancer Therapy (Humana Press 2001); the complete disclosures of each of which is hereby incorporated by reference.

Two general approaches may be used. In the first approach, extracellular MMP cleaves the TAT-TLK1B fusion protein at an MMP cleavage-sensitive motif between the TAT domain and the TLK1B domain. Bereft of the TAT cell-penetrating domain, the TLK1B is not taken up by cancer cells in significant concentrations. Extracellular cleavage of the protein at the tumor cell surface or within its microenvironment inhibits TLK1B penetration into cancer cells.

In the second approach, the MMP cleavage-sensitive motif is positioned within the TLK1B molecule itself. When the molecule is cleaved by MMP (whether on the surface of a cancer cell or in the vicinity of the cancer cell), the TLK1B molecule loses its protective function.

It is preferred that one approach or the other be used within a particular construct; it is not preferred that both approaches be employed within the same construct. The first approach renders normal cells better able to minimize the side effects of the principal therapy. The second approach serves the dual purposes of rendering normal cells better able to minimize side effects, and also sensitizing cancer cells, making them less able to withstand the therapy. In the second approach, MMP-induced cleavage within TAT-fused TLK1B results in truncated TLK1B molecules lacking the C-terminal kinase domain. TLK1 is known to dimerize, and to autophosphorylate to activate kinase activity. Inside cancer cells the truncated TLK1B molecules will likely dimerize with intracellular TLK1 produced by the cell itself, preventing autophosphorylation and resulting in a non-functional enzyme molecule. By effectively sequestering TLK1 monomers that the cell would otherwise use to produce functional dimeric enzymes, the effective concentration of functional TLK1 in the cancer cells declines, and hence the cancer cells are actually sensitized to the effects of radiation or chemotherapy. A multiplier in the therapeutic ratio is therefore expected—not only do normal cells become more resistant to side effects from the principal therapy, but the cancer cells become sensitive to the principal therapy.

The two approaches are illustrated schematically in FIGS. 2A and 2B. Abbreviations used in these figures: MS=MT1-MMP-sensitive site. His=Histidine 6×. TAT=11 amino acid HIV-TAT protein transduction domain. HA=Hemagglutinin tag. TLK-N=N-terminal TLK1B kinase domain. TLK-C=TLK1B C-terminal kinase domain. TLK-DN=dominant-negative TLK1B. “No radioresistance” in FIG. 2A means that the cancer cells remain more-or-less unaffected by the transduced protein, while normal cells are better protected. “Radiosensitized” in FIG. 2B means that the cancer cells are actually more susceptible to the radiation-induced lethality, while normal cells are protected.

MT1-MMP is membrane bound, and it is required for cleavage of pro MMP-2 to become the active MMP2 enzyme. Cleavage sequences that are sensitive to MT1-MMP or to MMP-2 may be used in the present invention. Most tumors of epithelial origin (e.g., HNSCC, breast, prostate, etc) overexpress MMPs. MMP expression is also related to tumor aggressiveness and metastasis. Cleavage site motifs identified by Turk (2001) show similarity between MMP2 and MT1-MMP sites. The designed sequence (VPLSLRSG, SEQ ID NO: 1) will likely be recognized by both MMPs.

The data showed that the novel approach successfully permits entry of functional protein (TAT-MS-TLK1B) into normal cells, but not in cancerous cells. Cells subjected to radiotherapy will, therefore, show differential responses to radiotherapy; normal cells will have better survival rates than cancer cells. Future tests will confirm that the novel approach has similar effects when used in conjunction with genotoxic chemotherapies, such as therapies using topoisomerase inhibitors (e.g., adriamycin, daunorubicin, camptothecin, etoposide, irinotecan, topotecan, and the like), and cross-linking and alkylating agents (e.g., cisplatin, carboplatin, cyclophosphamide, chlorambucil, and the like).

Where prior approaches have used MMP-sensitive sites to cleave a pro-toxin and thereby selectively activate a toxin in the vicinity of cancerous cells, the novel approach instead uses an MMP-sensitive site to cleave and selectively de-activate a protective compound at the surface of cancerous cells or in their vicinity. To my knowledge, such an approach has not previously been reported.

An alternative embodiment exploits the abundant expression of furin in cancer cells. Intracellular cleavage of TLK1B by furin is used to sensitize tumors to radiation or chemotherapy. Furin is overexpressed in many cancer cells. Furin cleaves pro-MT1-MMP into active MT1-MMP in the trans-Golgi network. The furin cleavage recognition sequence present at the C-terminal end of the propeptide is RRKR↓Y (SEQ ID NO: 5). (↓ indicates the cleavage site)

Proprotein convertases such as furin catalyze the proteolytic maturation of a protein precursor to a functionally active protein. Furin, an intracellular endoprotease, targets cleavage sites in the precursors of blood clotting factors (e.g., von Willibrand factor), serum proteins (e.g., albumin), extracellular matrix proteins, extracellular proteases (e.g., MT1-MMPs), receptors (e.g., integrins, insulin-like growth factor 1 (IGF1)), hormones (e.g., parathyroid hormone) and cytokines (e.g., TGF beta 1). Many, but not all cleavage recognition sequences conform to the motif RX(R/K/X)R↓. The furin-sensitive site within pro-MT1-MMP, RRKR (SEQ ID NO: 81), follows this pattern, but those of serum albumin, VFRR (SEQ ID NO: 82), and C-type natriuretic peptide, RLLR (SEQ ID NO: 83) do not. Furin is overexpressed in many cancer cells.

Additional steps will likely be needed to deliver the proteins of this invention to deeper tissues. For deeper penetration of TAT-fusion proteins to the basal layer of the epithelium or to oral mucosa, for example, mechanical or chemical facilitators otherwise known in the art (e.g., microneedles, ultrasound, or penetration enhancers) may be used. When such a facilitator is used, there could be a possibility that the recombinant MS-sensitive TAT-TLK1B could enter cells non-selectively, directly through “pores” generated in cell membranes by such facilitators. An alternative embodiment of the invention, one that helps safeguard against the possible presence of functional TLK1B molecules inside cancer cells, employs a furin cleavage sequence—either alone or in conjunction with an MS site—incorporated into the TAT-TLK1B fusion protein. Intracellular cleavage of the TLK1B protein then occurs predominantly in furin-overexpressing cancer cells, to deactivate the functional molecule in the cancer cells. In addition to the furin cleavage sites mentioned in the previous paragraph, other furin cleavage sites from human protein precursors include the following: Integrin alpha 4 precursor furin cleavage site: HVISKR (SEQ ID NO:84); Pro-parathyroid hormone, KSVKKR (SEQ ID NO:85); Vascular endothelial growth factor C precursor, HSIIRR (SEQ ID NO:86); Albumin precursor, RGVFRR (SEQ ID NO:87); and Furin self-cleavage site, RGVTKR (SEQ ID NO:88).

A major limitation of many prior cancer therapies has been the dosage that normal tissues can withstand (whether radiation or chemotherapy). The novel approach selectively protects normal tissues without substantially compromising tumor control. The novel approach should allow the use of higher dosages of chemotherapies, radiotherapy, or both than has previously been feasible, leading to improved clinical outcomes for patients.

Because the kinase activity of TLK1B is an important contributor to cellular response to genotoxic stresses, inserting an MMP-sensitive site (MS) within TLK1B causes the TLK1B to express in a dominant negative fashion to radiosensitize tumors (See, e.g., FIG. 2B).

The data have demonstrated the in vitro cleavage of MS-incorporated TAT-TLK1B. The presence of an MT1-MMP cleavage site (MS) between TAT and TLK1B greatly increased the sensitivity of TAT-MS-TLK1B to cleavage as compared to TAT-TLK1B (i.e., with no MS site) when incubated with recombinant MT1-MMP catalytic enzyme.

The data have also demonstrated the in vivo transduction of cleaved TAT-MS-TLK1B into HNSCC cells (cell line SCC40). Increased expression of MMP by SCC40 cleaved TAT-MS-TLK1B and restricted entry of TLK1B into cells.

### BRIEF DESCRIPTION OF THE DRAWINGS

FIG. 1 depicts a schematic representation of TLK1 mRNA and TLK1B translation from mRNA.

FIG. 2A depicts schematically cancer cells remaining more-or-less unaffected by a transduced protein that protects normal cells from cancer therapy.

FIG. 2B depicts schematically cancer cells being selectively sensitized to a cancer therapy, while normal cells are protected.

FIG. 3 depicts anti-His immunoblotting of TAT-TLK1B or TAT-MS-TLK1B, reacted with the MT1-MMP catalytic domain as a function of time.

FIG. 4A depicts the results of immunoblotting cell lysates with anti-(MT1-MMP catalytic domain) antibody.

FIG. 4B depicts results of immunoblotting cell lysates with horseradish peroxidase-conjugated anti-hemagglutinin.

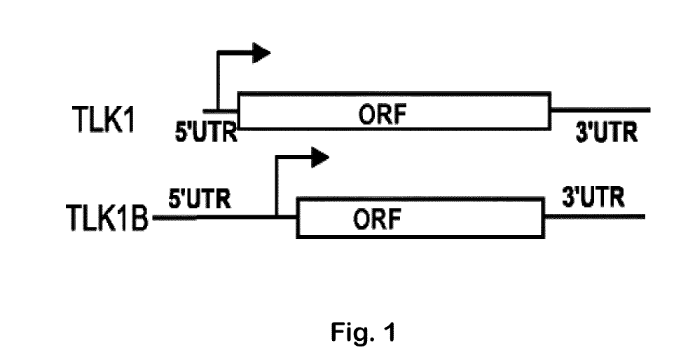

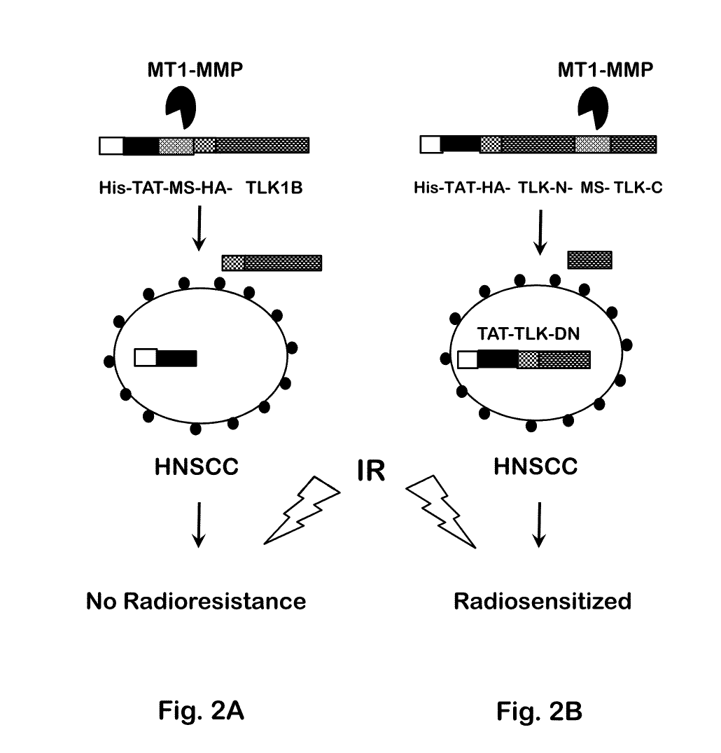

Escalating the radiation dose (or the chemotherapy dose) would clearly help in eradicating tumors, but there are limits on the dosage that may be tolerated without an increased chance of severe radiation damage (or severe chemotherapy damage) to normal tissues. The cell-permeable protein TAT-TLK1B substantially protects the viability and function of cells, for example salivary cells, in irradiated animals.

One example where the novel approach is expected to be particularly beneficial is for patients undergoing emergency head and neck radiation, where immediate expression of TLK1B is necessary to protect salivary glands. Another use is as a topical treatment for oral mucositis, targeting the protein to oral mucosal cells. The original objective was to deliver TAT-TLK1B as a safe therapeutic for mitigation of oral complications, but it can also be used to mitigate the effects of cancer therapy for other tissues and organs, for example the gastrointestinal tract; or to protect the skin from radiation dermatitis or radiation “burns.”

The present invention may be practiced as a combination therapy with forms of radiotherapy or chemotherapy otherwise known in the art. Suitable chemotherapeutic agents include naturally occurring, semi-synthetic or synthetic therapeutic agents and can be any available chemotherapeutic or naturally occurring, semi-synthetic or synthetic therapeutic agent, and more particularly the chemotherapeutic agents which are commonly used for the treatment of cancer.

Other forms of cancer besides head and neck cancers may be treated in accordance with this invention, including solid tumors, such as urogenital cancers (such as prostate cancer, renal cell cancers, bladder cancers), gynecological cancers (such as ovarian cancers, cervical cancers, endometrial cancers), lung cancer, gastrointestinal cancers (such as colorectal cancers, pancreatic cancer, gastric cancer, esophageal cancers, hepatocellular cancers, cholangiocellular cancers), malignant mesothelioma, breast cancer, malignant melanoma or bone and soft tissue sarcomas, and cancers of the eye and skin (such as melanoma); and hematologic neoplasias, such as multiple myeloma, acute myelogenous leukemia, chronic myelogenous leukemia, myelodysplastic syndrome and acute lymphoblastic leukemia.

The beneficial efficacy of the methods in accordance with the invention are mainly based on the additive and synergistic effects of the combined treatment, or to an improved tolerability of the treatment by the patient due to the administration of higher doses of radiotherapy or chemotherapeutic agents than would otherwise be practical.

In another embodiment, the present invention also relates to a pharmaceutical combination preparation kit for the treatment of cancers, characterized in that the recombinant protein is contained within a first compartment; and a chemotherapeutic or naturally occurring, semi-synthetic or synthetic therapeutic agent is contained within a second compartment, such that the administration to a patient in need thereof can be simultaneous, separate, or sequential, said combination preparation kit being optionally further adapted for a co-treatment with radiotherapy or radio-immunotherapy.

In another embodiment, the present invention thus also provides for the use of the recombinant protein in combination with a chemotherapeutic or naturally occurring, semi-synthetic or synthetic therapeutic agent, or adapted for a co-treatment with radiotherapy or radio-immunotherapy; for the manufacture of a pharmaceutical combination preparation for the treatment of cancer.

As used in the specification and claims, an “effective amount” of a therapeutic agent, co-administered agent, or radiotherapy refers to an amount that is effective to achieve a therapeutic effect when used in combination; for example, an effective amount to inhibit or reduce the growth of one or more tumors. It is understood that “an effective amount” or “a therapeutically effective amount” varies from subject to subject, due to variations in metabolism of the compound administered, the age, weight, and general condition of the subject, the condition being treated, the severity of the condition being treated, and the judgment of the prescribing physician.

As used in the specification and claims, a cancerous or tumor cell “overexpresses” a protein if the in vivo level of expression of the protein in the tumor cell is at least 50% greater than, preferably at least 100% greater than, and more preferably 150% or more greater than the level of expression of the same protein in normal, noncancerous cells of the same lineage in the same organism.

These and other features are explained more fully in the embodiments described below. It should be understood that in general the features of one embodiment also may be used in combination with features of another embodiment and that the embodiments are not intended to limit the scope of the invention.

It is to be understood that other embodiments may be utilized and changes may be made without departing from the scope of the present invention. Also, it is to be understood that the phraseology and terminology used herein are for the purpose of description and should not be regarded as limiting. The use of terms such as “including,” “comprising,” “containing,” “having,” variations thereof, and the like are intended to encompass the items specifically mentioned and equivalents thereof, as well as the possibility that additional components might also be present.

As used in the specification and the claims, the singular forms “a”, “an,” and “the” can include plural referents unless the content clearly dictates otherwise. Thus, for example, reference to “a component” can include a combination of two or more components; a reference to “containers” can include individual containers, and so forth.

As used in the specification and claims, the terms “administer”, “administered”, and “administration” of the various substances denote providing an amount of a substance by any suitable means, including topical delivery, injection, oral administration, a parenterally administered prolonged release delivery system (e.g., pellet, liquid depot, suppository or the like), or by continuous dosing (e.g., by an infusion pump) of the substance, delivered as a one-time administration, periodically, or continuously.

As used in the specification and claims, “ameliorating,” “mitigating,” “lessening of the symptoms” or the like for a particular symptom, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

As used in the specification and claims, to “not substantially inhibit” the activity of a kinase means that the presence of an exogenous sequence in a the protein, such as an intact MMP cleavage site, either leaves the kinase activity of the protein unaltered as compared to the native or wild-type kinase; or it enhances the kinase activity of the protein; or it reduces the kinase activity of the protein by a small amount—by less than 25%, preferably by less than 10%, more preferably by less than 5%. The “kinase activity” is defined as the rate at which the kinase phosphorylates its substrate; in the case of the TLKs, the rate of phosphorylation of its substrate, histone H3.

As used in the specification and claims, to “substantially reduce” the activity of a kinase means that, following cleavage at a proteolysis site within the kinase, the domain either loses all kinase activity towards its substrate (as defined in the previous paragraph); or, if it retains any kinase activity, that activity is reduced by a large amount—by more than 50%, preferably more by than 80%, more preferably by more than 95%.

The term “cancer” includes solid tumors such as those of the breast, ovary, prostate, lung, kidney, stomach, colon, testes, head and neck, pancreas, brain, melanoma, and other tumors of tissue organs and cancers of the blood cells, such as lymphomas and leukemias, including acute myelogenous leukemia, chronic lymphocytic leukemia, T cell lymphocytic leukemia, and B cell lymphomas.

The term “DNA-Damaging Treatment” includes treatments that cause DNA damage and include those such as gamma-rays, X-rays, particle radiation (e.g., proton beam, neutron radiation, etc.), alpha particle therapy (such as immunotargeted therapy with ²¹²Pb) or the directed delivery of radioisotopes to tumor cells. It is most likely that these factors inflict a broad range of damages on DNA and affect DNA replication, gene expression and the assembly and maintenance of chromosomes. Other agents that damage DNA include compounds also described as “Chemotherapeutic agents.” Agents such as cisplatin or other DNA alkylating drugs may be used. Agents that damage DNA also include compounds that interfere with DNA replication, mitosis, and chromosomal segregation. Examples of such compounds includes etoposide, camptothecin, doxorubicin, and the like. Photodynamic therapy that may also be used in conjunction with the present invention; in photodynamic therapy cell toxicity is generating by ultraviolet exposure of a photosensitizer such as aminolevulinic acid, Silicon Phthalocyanine Pc 4, m-tetrahydroxyphenylchlorin, mono-L-aspartyl chlorin e6, Allumera, Photofrin, Visudyne, Levulan, Foscan, Metvix, Hexvix, Cysview, Laserphyrin, Antrin, Photochlor, Photosens, Photrex, Lumacan, Cevira, Visonac, BF-200 ALA, or Amphinex.

The term “inhibiting” includes preventing, slowing, or reversing the development of a cancer.

The term “pharmaceutically” or “pharmacologically acceptable” refers to compositions that do not produce adverse reactions when administered to an animal or a human.

The term “pharmaceutically acceptable carrier” includes biocompatible solvents or dispersion media including, but not limited to, water, ethanol, polyols (for example, glycerol, propylene glycol, and liquid polyethylene glycol, and the like), mixtures thereof, and vegetable oils, coatings, isotonic and absorption delaying agents, liposomes, commercially available cleansers, and the like. Supplementary bioactive ingredients also can be incorporated into such carriers.

The term “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in the art using known methods for such derivatization. The compounds produced may be administered to animals or humans without substantial toxic effects and either are pharmaceutically active or are prodrugs. In addition, a single-isomer formulation of a racemic compound, or vice versa, is also a “pharmaceutically acceptable derivative.”

Pharmaceutically acceptable salts include, but are not limited to, amine salts, including but not limited to N,N′-dibenzylethylenediamine, chloroprocaine, choline, ammonium, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium salts; alkali earth metal salts, such as but not limited to barium, calcium and magnesium salts; transition metal salts, such as but not limited to zinc salts; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. The pharmaceutically acceptable salts also include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two.

Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or 1 to about 2, 3 or 4, solvent or water molecules.

A “prodrug” refers to a compound or agent that is converted into the parent drug in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be generated or regenerated upon in vivo administration. In one embodiment, the prodrug is designed to alter the absorption and/or the transport characteristics of a drug, or to alter other characteristics or properties of a drug.

“Protecting”, as used in the context of ionizing radiation or chemotherapy, is meant to refer to any measurable or otherwise observable reduction in one or more of the harmful effects of ionizing radiation or chemotherapy. Such reduction in a harmful effect can be ascertained directly, e.g., by monitoring DNA or other cellular changes, or indirectly, by qualitatively or quantitatively evaluating a subject's symptoms resulting from ionizing radiation exposure. The protection need not be, and in many cases will not be a complete (100%) reduction in the harmful effects of the treatment. Such reduction can be observed in terms of the severity of the harmful effect, the duration of the harmful effect, or both; and it can be qualitative or quantitative. Examples of harmful effects from which a subject can be protected in accordance with the method of the present invention include: radiation sickness, hair loss (alopecia), weakness, fatigue, nausea, vomiting, diarrhea, skin burns, gastrointestinal tract bleeding, mucous membrane bleeding, gastrointestinal sloughing, oral mucosal sloughing, genetic defects, hematopoietic and/or immunocompetent cell destruction, sterility, bone marrow cancer and other kinds of cancer, premature aging, death, venoocclusive disease of the liver, chronic vascular hyperplasia of cerebral vessels, cataracts, and pneumonites.

Radiation therapy, radio-immunotherapy or pre-targeted radioimmunotherapy are used for the treatment of cancers. “Radiotherapy”, or radiation therapy, means the treatment of cancer with ionizing radiation. Ionizing radiation deposits energy that injures or destroys cells in the area treated (the target tissue) by damaging genetic material, making it impossible for these cells to continue to grow or metabolize. Radiotherapy may be used to treat localized solid tumors, such as cancers of the skin, tongue, larynx, brain, breast, lung, uterus, cervix, or head and neck. It can also be used to treat leukemia and lymphoma. One type of radiation therapy commonly used involves photons, e.g. X-rays. Depending on the amount of energy they possess, x-rays can be used to destroy cancer cells on the surface of or deeper in the body. The higher the energy of the x-ray beam, the deeper the x-rays can go into the target tissue. Linear accelerators and betatrons are machines that can be used to produce x-rays of increasingly greater energy. The use of machines to focus radiation (such as x-rays) on a cancer site is called external beam radiotherapy Gamma rays are another form of photons used in radiotherapy Gamma rays are produced spontaneously as certain elements (such as radium, uranium, and cobalt 60) release radiation as they decay. Another technique for delivering radiation to cancer cells is to place radioactive implants directly in a tumor or body cavity. This is called internal radiotherapy. Brachytherapy, interstitial irradiation, and intracavitary irradiation are types of internal radiotherapy. In this treatment, the radiation dose is concentrated in a small area, and the patient typically stays in the hospital for a few days. Internal radiotherapy is frequently used for cancers of the tongue, uterus, and cervix. A further technique is intra-operative irradiation, in which a large dose of external radiation is directed at the tumor and surrounding tissue during surgery. Another approach is particle beam radiation therapy. This type of therapy differs from photon radiotherapy in that it involves the use of fast-moving subatomic particles to treat localized cancers. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation. Hyperthermia, the use of heat, may be used for sensitizing tissue to radiation. Another option involves the use of radio-labeled antibodies (including targeted alpha particle therapy (high LET radiation) using ²¹²Pb conjugates) to deliver doses of radiation directly to the cancer site (radio-immunotherapy). Another approach is light-targeted activation of a photosensitizer drug that generates reactive radicals within cells (photodynamic therapy).

As used herein, the term “subject” is used to mean an animal, preferably a mammal, including a human or non-human mammal. The terms patient and subject may be used interchangeably. The subject is suffering from cancer.

The term “tousled-like kinases” or “TLK” refer to nuclear serine/threonine kinases that are involved in the regulation of chromatin assembly. They are rapidly and transiently inhibited by phosphorylation following a DNA double-stranded break during S-phase. The TLK1 protein contains a protein kinase ATP-binding motif. TLK1 is expressed in almost all tissues. The 787-amino acid protein TLK1 has a 5-domain structure, with N-terminal nuclear localization signals followed by a nucleotide binding motif, and a single catalytic domain near the C terminus. It shares 86% sequence identity with TLK2 overall, and 94% identity in the catalytic region. TLK1 localizes in the nucleus.

DNA-damaging agents or factors used in conjunction with the recombinant proteins of the invention may be any chemical compound or treatment method that induces DNA damage when applied to a cell. Such agents and factors include radiation that induce DNA damage, such as gamma rays, X-rays, and the like. A variety of chemical compounds, also described as “Chemotherapeutic agents” function to induce DNA damage, all of which are included to be of use in the combined treatment methods disclosed herein. Chemotherapeutic agents include by way of example alkylating agents (e.g. cis-diamine dichloroplatinum (CDDP) or melphalan), agents that interfere with DNA replication, mitosis, and chromosomal segregation (e.g. etoposide (VP-16), camptothecin and adriamycin, also known as doxorubicin), radiomimetic agents (e.g. bleomycin).

Treatment regimens may vary as well, and may depend on the type of ionizing damage or exposure, location of damage or exposure, disease progression, and health and age of the patient. Certain types of cancer require more aggressive treatment, while at the same time, certain patients cannot tolerate more taxing protocols. The clinician will be best suited to make such decisions based on the known efficacy and toxicity (if any) of the therapeutic formulations.

The treatment should be administered far enough in advance of the therapeutic radiation or chemotherapy that the administered protein reaches the cells of the subject in sufficient concentration to exert a protective or sensitizing effect on the cells, or both, as appropriate. The treatment may be administered as much as about 24 hours, preferably no more than about 18 hours, prior to administration of the principal therapy. In one embodiment, the treatment is administered at least about 6-12 hours before administration of the principal therapy. Most preferably, the treatment is administered once at about 18 hours and again at about 6 hours before the principal therapy.

In specific embodiments, the treatment is administered in a single dose or multiple doses. The single dose may be administered daily, or multiple times a day, or multiple times a week. In a further embodiment, the treatment is administered in a series of doses. The series of doses may be administered daily, or multiple times a day, weekly, or multiple times a week.

The improvement is any observable or measurable change for the better. The composition and the method of treatment of this invention may decrease the mortality of subjects exposed to damaging radiation or chemotherapy. In other aspects, the composition of this invention is administered in an effective amount to decrease, reduce, inhibit, prevent or eliminate damage to, and the loss of function of the salivary glands or xerostomia.

Examples of DNA-damaging agents that may be used in combination with compounds of this invention include, but are not limited to platinating agents, such as carboplatin, nedaplatin, satraplatin and other derivatives; topoisomerase inhibitors, such as topotecan, irinotecan/sn38, rubitecan and other derivatives; antimetabolites, such as folic family (methotrexate, pemetrexed and relatives); purine antagonists and pyrimidine antagonists (thioguanine, fludarabine, cladribine, cytarabine, gemcitabine, 6-mercaptopurine, 5-fluorouracil (5fu) and relatives); alkylating agents, such as nitrogen mustards (cyclophosphamide, melphalan, chlorambucil, mechlorethamine, ifosfamide and relatives); nitrosoureas (eg carmustine); triazenes (dacarbazine, temozolomide); alkyl sulphonates (eg busulfan); procarbazine and aziridines; antibiotics, such as hydroxyurea, anthracyclines (doxorubicin, daunorubicin, epirubicin and other derivatives); anthracenediones (mitoxantrone and relatives); streptomyces family (bleomycin, mitomycin c, actinomycin); abarelix (plenaxis depot); aldesleukin (prokine); aldesleukin (proleukin); alemtuzumabb (campath); alitretinoin (panretin); allopurinol (zyloprim); altretamine (hexylen); amifostine (ethyol); anastrozole (arimidex); arsenic trioxide (trisenox); asparaginase (elspar); azacitidine (vidaza); bevacuzimab (avastin); bexarotene capsules (targretin); bexarotene gel (targretin); bleomycin (blenoxane); bortezomib (velcade); busulfan intravenous (busulfex); busulfan oral (myleran); calusterone (methosarb); capecitabine (xeloda); carboplatin (paraplatin); carmustine (bcnu, bicnu); carmustine (gliadel); carmustine with polifeprosan 20 implant (gliadel wafer); celecoxib (celebrex); cetuximab (erbitux); chlorambucil (leukeran); cisplatin (platinol); cladribine (leustatin, 2-cda); clofarabine (clolar); cyclophosphamide (cytoxan, neosar); cyclophosphamide (cytoxan injection); cyclophosphamide (cytoxan tablet); cytarabine (cytosar-u); cytarabine liposomal (depocyt); dacarbazine (dtic-dome); dactinomycin, actinomycin d (cosmegen); darbepoetin alfa (aranesp); daunorubicin liposomal (danuoxome); daunorubicin, daunomycin (daunorubicin); daunorubicin, daunomycin (cerubidine); denileukin diftitox (ontak); dexrazoxane (zinecard); docetaxel (taxotere); doxorubicin (adriamycin pfs); doxorubicin (adriamycin, rubex); doxorubicin (adriamycin pfs injection); doxorubicin liposomal (doxil); dromostanolone propionate (dromostanolone); dromostanolone propionate (masterone injection); Elliott's b solution (Elliott's b solution); epirubicin (ellence); epoetin alfa (epogen); erlotinib (tarceva); estramustine (emcyt); etoposide phosphate (etopophos); etoposide, vp-16 (vepesid); exemestane (aromasin); filgrastim (neupogen); floxuridine (intraarterial) (fudr); fludarabine (fludara); fluorouracil, 5-fu (adrucil); fulvestrant (faslodex); gefitinib (iressa); gemcitabine (gemzar); gemtuzumab ozogamicin (mylotarg); goserelin acetate (zoladex implant); goserelin acetate (zoladex); histrel in acetate (histrelin implant); hydroxyurea (hydrea); ibritumomab tiuxetan (zevalin); idarubicin (idamycin); ifosfamide (ifex); imatinib mesylate (gleevec); interferon alfa 2a (roferon a); interferon alfa-2b (intron a); irinotecan (camptosar); lenalidomide (revlimid); letrozole (femara); leucovorin (wellcovorin, leucovorin); leuprolide acetate (eligard); levamisole (ergamisol); lomustine, ccnu (ceebu); meclorethamine, nitrogen mustard (mustargen); megestrol acetate (megace); melphalan, 1-pam (alkeran); mercaptopurine, 6-mp (purinethol); mesna (mesnex); mesna (mesnex tabs); methotrexate (methotrexate); methoxsalen (uvadex); mitomycin c (mutamycin); mitotane (lysodren); mitoxantrone (novantrone); nandrolone phenpropionate (durabolin-50); nelarabine (arranon); nofetumomab (verluma); oprelvekin (neumega); oxaliplatin (eloxatin); paclitaxel (paxene); paclitaxel (taxol); paclitaxel protein-bound particles (abraxane); palifermin (kepivance); pamidronate (aredia); pegademase (adagen (pegademase bovine)); pegaspargase (oncaspar); pegfilgrastim (neulasta); pemetrexed disodium (alimta); pentostatin (nipent); pipobroman (vercyte); plicamycin, mithramycin (mithracin); porfimer sodium (photofrin); procarbazine (matulane); quinacrine (atabrine); rasburicase (elitek); rituximab (rituxan); sargramostim (leukine); sargramostim (prokine); sorafenib (nexavar); streptozocin (zanosar); sunitinib maleate (sutent); talc (sclerosol); tamoxifen (nolvadex); temozolomide (temodar); teniposide, vm-26 (vumon); testolactone (teslac); thioguanine, 6-tg (thioguanine); thiotepa (thioplex); topotecan (hycamtin); toremifene (fareston); tositumomab (bexxar); tositumomab/i-131 tositumomab (bexxar); trastuzumab (herceptin); tretinoin, atra (vesanoid); uracil mustard (uracil mustard capsules); valrubicin (valstar); vinblastine (velban); vincristine (oncovin); vinorelbine (navelbine); zoledronate (zometa) and vorinostat (zolinza).

The TLK kinase modulators or pharmaceutical salts thereof may be formulated into pharmaceutical compositions for administration to animals or humans. These pharmaceutical compositions, which comprise an amount of the TLK inhibitor effective to treat or prevent the diseases or conditions described herein and a pharmaceutically acceptable carrier, are another embodiment of the present invention.

The exact amount of compound required for treatment will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

SEQ ID NO: 2 is the sequence for the TAT-TLK1B fusion protein, without the MS cleavage site. The first 56 amino acids at the amino terminus of the protein (from MHH . . . to . . . SVD) are from the pET 30b+ vector, which includes the His-tag and S-tag.

### EXAMPLE 1

Except as otherwise noted, all recombinant proteins were produced using methods that are otherwise standard and well-known in the art. The recombinant proteins were expressed in BL21 (DE3)pLysS (E. coli), and were isolated by Ni-NTA agarose affinity chromatography, followed by FPLC purification.

### EXAMPLE 2

The initial attempt was unsuccessful, to make a working construct embodying the novel complementary therapy approach. The initial construct lacked the kinase activity needed for the intended purpose.

The secondary and tertiary structure of TLK1B had not previously been reported. The likely structure of TLK1B was predicted by homology modeling, using information obtained from the SWISS-PROT and RCSB Protein databases. The serine/threonine kinase domain of TLK1B was aligned to proteins with similar conserved features. Sequence alignment with cyclin dependent kinase 2 (cdk2), a serine/threonine kinase, suggested a possible structure of the TLK1B kinase region (data not shown).

Important elements of TLK1B kinase domain were modeled, including presumptive substrate binding sites, a presumptive ATP-binding pocket, and a presumptive activation loop (data not shown). An MT1-MMP-cleavage sequence (MS) was initially inserted downstream of these elements, within a presumptive loop region. I expected this location to be one for which the inserted sequence would least affect the structure or activity of TLK1B, due to the flexibility that the presumptive loop region was expected to have. An initial TAT-TLK-MS sequence was constructed accordingly, with the MS sequence in the presumptive loop element, and evaluated the efficacy of the recombinant protein. It was surprising and disappointing to find that the recombinant protein, although sensitive to cleavage by the catalytic MT1-MMP protein as designed, would not phosphorylate the TLK substrate, histone H3 Ser-10. Surprisingly, inserting the MS site into a presumptively “flexible” loop region resulted in loss of kinase activity for the TAT-TLK-MS protein (data not shown).

The initial attempt, inserting the MS site into a rationally-chosen location, did not work.

SEQ ID NO: 3 is the sequence for the TAT-TLK-MS fusion protein. The first 56 amino acids at the amino terminus of the protein (from MHH . . . to . . . SVD) are from the pET 30b+ vector, which includes the His-tag and S-tag. The 8-amino acid sequence VPLSLRSG (SEQ ID NO: 1) appearing towards the carboxy terminus is the MT1-MMP cleavage site.

### EXAMPLE 3

In the next attempt, the MS was inserted 5′ to the kinase domain of TLK1B. The recombinant TAT-TL-MS-K protein was produced, purified, and the activity of the protein was analyzed in vitro. This time the TAT-TL-MS-K retained the native kinase activity, as indicated by phosphorylation of histone H3 (data not shown). Activity of the TAT-TL-MS-K was monitored indirectly by evaluation of histone H3 Seri 0 phosphorylation. Recombinant TAT-TLK1B or TAT-TL-MS-K was reacted with histone H3 at ratios of 1:10, 1:5, or 1:1 in a kinase buffer at 30° C. Reactions were loaded onto SDS-PAGE gels, and immunoblotted against anti-Ser10 H3 (Millipore). The TAT-TL-MS-K was more sensitive to MT1-MMP cleavage than was TAT-TLK1B (data not shown). The functional, MMP-sensitive TAT-TL-MS-K will be used to confirm transduction in normal and cancer cells, and the ability of TAT-TL-MS-K to sensitize cancer cells such as head and neck cancer cells to radiation.

SEQ ID NO: 4 is the sequence for the TAT-TL-MS-K fusion protein. The first 56 amino acids at the amino terminus of the protein (from MHH . . . to . . . SVD) are from the pET 30b+ vector, which includes the His-tag and S-tag. The 8-amino acid sequence VPLSLRSG (SEQ ID NO: 1) appearing roughly in the middle of the sequence is the MT1-MMP cleavage site.

### EXAMPLE 4

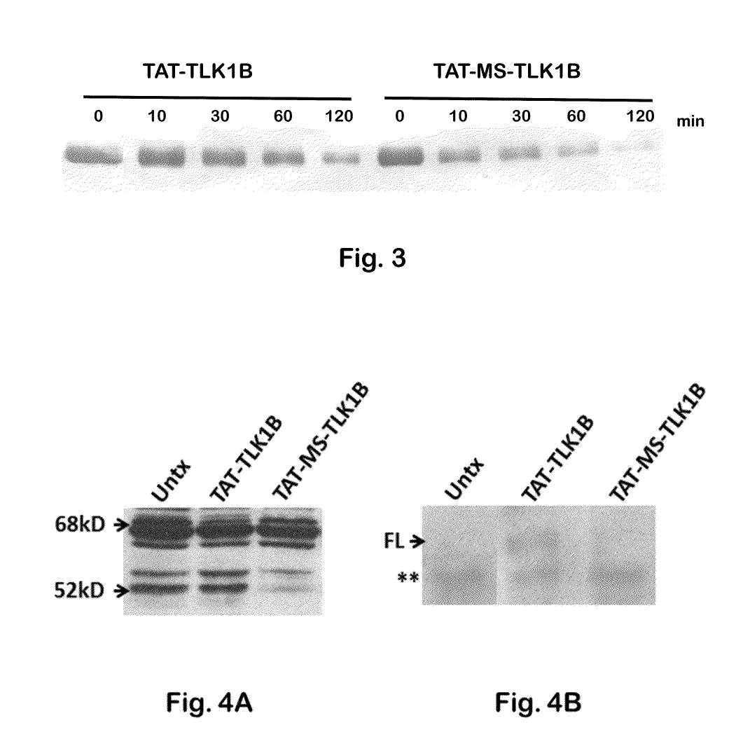

FIG. 3 depicts anti-His immunoblotting of TAT-TLK1B or TAT-MS-TLK1B, reacted with the MT1-MMP catalytic domain (EMD Millipore) at 22° C., as a function of time. 1.5 μg TAT-TLK1B, TAT-MS-TLK1B or TAT-TL-MS-K was reacted with 0.02 mU MT1-MMP, catalytic domain (Calbiochem) in reaction buffer containing 50 mM Tris pH 7.5, 150 mM NaCl, 5 mM CaCl₂. The reactions were incubated at 22° C. for the indicated times (10, 20, 30, 60, or 120 minutes). After 120 minutes, essentially all the TAT-MS-TLK1B had been cleaved, while the TAT-TLK1B still showed substantial activity. Note that the TAT-TLK1B was also cleaved by the enzyme, although not as rapidly. Although MT1-MMP has a preference for the preferred cleavage site motif, that preference is neither strict nor absolute. Thus other proteins will be cleaved as well; what is important is the relative sensitivity of MS-containing proteins to MT1-MMP cleavage.

### EXAMPLE 5

HNSCC cells (cell line SCC40) were cultured on rat tail type I collagen plates in DMEM/0.1% BSA for 3 days before addition of 40 μg TAT-TLK1B or TAT-MS-TLK1B. Control cells (Untreated cells, Untx) were treated with an equal volume of OPTI-MEM™ reduced serum medium—a modification of Eagle's Minimum Essential Media, buffered with HEPES and sodium bicarbonate, and supplemented with hypoxanthine, thymidine, sodium pyruvate, L-glutamine, trace elements, and growth factors. FIG. 4A depicts the results of immunoblotting the respective cell lysates with anti-(MT1-MMP catalytic domain) antibody (Millipore). The arrow at 68 kD corresponds to pro-MT1-MMP. The active MT1-MMP was 65 kD, and was seen in all three lanes. The 52 kD and 55 kD proteins were products of autocatalytic cleavage of MT1-MMP. The autoproteolytic products declined sharply in the presence of the TAT-MS-TLK1B. Active MT1-MMP is membrane-bound, and is responsible for cleaving and activating pro-MMP-2 to active MMP-2. Active MT1-MMP can also undergo autocatalytic processing. The presence of a substrate such as TAT-MS-TLK1B or TAT-TL-MS-K will decrease the MT1-MMP autocatalysis. Suppression of MT1-MMP autocatalysis suggests that the recombinant MMP-sensitive TAT-fusion proteins are being cleaved by MT1-MMP instead. In other words, the observed decrease in MT1-MMP auto-cleavage is indirect evidence that the recombinant TAT-MS-fusion protein is being cleaved as designed. The more direct evidence was the western blot of cell lysates, which showed failure of TLK1B transduction following MT1-MMP cleavage in the HNSCC cell line SCC40.

FIG. 4B depicts results of immunoblotting the respective cell lysates with horseradish peroxidase-conjugated anti-hemagglutinin (Sigma) 6 hours after protein transduction. FL: full-length recombinant TAT-fusion protein; **—non-specific cellular protein. The HNSCC cell line, SCC40, when grown on collagen plates, demonstrated the expression of MT1-MMP (FIG. 4A), and decreased translocation of TAT-MS-TLK1B across the cell membrane (FIG. 4B). The western blot of FIG. 4B shows that TLK1B was selectively excluded inside the SCC40 cancer cells only when the fusion protein included an MMP-cleavage site. I.e., TAT-TLK1B efficiently transduced into SSC40 cancer cells, but TAT-MS-TLK1B did not transduce efficiently into the SSC40 cancer cells. The label ** indicates a non-specific protein band seen in all lanes, suggestive of equal total protein loading.

Compounds used in the present invention may be administered to a patient by any suitable means, including topical, enteral, parenteral, intrapulmonary, and intranasal administration. Parenteral infusions include intramuscular, intravenous, intraarterial, intraperitoneal intradermal, subcutaneous, epidermal, intrathecal, intracerebral, or intraosseus administration. The compounds may also be administered transdermally, for example in the form of a slow-release subcutaneous implant. They may also be administered by inhalation.

Pharmaceutically acceptable carrier preparations include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

A compound in accordance with the present invention may be formulated into therapeutic compositions as pharmaceutically acceptable salts. These salts include acid addition salts formed with inorganic acids, for example hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

A method for controlling the duration of action comprises incorporating the active compound into particles of a polymeric substance such as a polyester, peptide, hydrogel, polylactide/glycolide copolymer, or ethylenevinylacetate copolymers. Alternatively, an active compound may be encapsulated in nanoparticles or microcapsules by techniques otherwise known in the art including, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

As used herein, an “effective amount” of a compound is an amount, that when administered to a patient (whether as a single dose or as a time course of treatment) inhibits or reduces the growth of targeted tumors to a clinically significant degree; or alternatively, to a statistically significant degree as compared to control. “Statistical significance” means significance at the P<0.05 level, or such other measure of statistical significance as would be used by those of skill in the art of biomedical statistics in the context of a particular type of treatment or prophylaxis.

As persons of skill in the art will recognize, the amino acids in a protein or peptide sequence can often be modified slightly without greatly affecting the properties of the protein or peptide. This invention relates not only to functional proteins and peptides as otherwise described herein, but also to proteins and peptides having modified sequences and still having the same or similar properties as described herein; and having 90% or greater, 91% or greater, 92% or greater, 93% or greater, 94% or greater, 95% or greater, 96% or greater, 97% or greater, 98% or greater, or 99% or greater sequence identity to the sequences disclosed herein. When amino acids are substituted, it is usually (but not invariably) preferred to make conservative substitutions (e.g., one acidic amino acid for another acidic amino acid, basic for basic, polar for polar, nonpolar for nonpolar, aromatic for aromatic). However, it can also be the case that certain nonconservative substitutions can sometimes be made without altering functionality, as can certain deletions and insertions. The degree of sequence identity can be determined by simple alignment based on programs known in the art, such as, for example, GAP and PILEUP by GCG, or the BLAST software available through the NIH internet site.

A cell-penetrating peptide that does not interrupt the functional portion of a protein should not be included when determining degree of sequence identity of the protein. Nor should the sequence of a cleavage site, such as an MMP cleavage site or furin cleavage site, be included when determining degree of sequence identity—even where the cleavage site is internal to the functional portion of the protein.

As used in the specification and claims, an overexpressed matrix metalloproteinase or an overexpressed furin “differentially causes” cleavage of a proteolytic cleavage site on tumor cells, inside tumor cells, or in the immediate environment of tumor cells to occur at a “substantially higher rate” than cleavage of the same proteolytic cleavage site on non-cancerous cells, inside non-cancerous cells, or in the immediate environment of non-cancerous cells if:

The ratio of the (cleavage rate on tumor cells, inside tumor cells, or in the immediate environment of tumor cells) to the (cleavage rate on non-cancerous cells, inside non-cancerous cells, or in the immediate environment of non-cancerous cells)

is 5:1 or greater, preferably 10:1 or greater, and more preferably 25:1 or greater.

Alternatively, without directly measuring or comparing these cleavage rates, such differential cleavage rates may be inferred where a clinically significant benefit is empirically observed in a cancer patient who receives the co-administration of a protein in accordance with this invention and a genotoxic principal therapy.

Those of skill in the art will appreciate that the co-administration of a protein in accordance with this invention and a genotoxic principal therapy does not necessarily mean that both are administered simultaneously. Although they can be administered simultaneously, in general it is preferred to administer the protein of this invention first, and then to administer the genotoxic principal therapy after a time, so that the protein of this invention can prime cells before they are subjected to the genotoxic therapy.

All references cited in this specification are hereby incorporated by reference in their entirety, as is the entire disclosure of provisional application 62/048,314. In the event of an otherwise irreconcilable conflict, the present specification shall control.

1. A recombinant protein comprising a cell penetrating peptide domain, a kinase domain, and at least one proteolytic cleavage site, wherein:
(a) said cell penetrating peptide domain, said kinase domain, and said matrix metalloproteinase cleavage site are covalently linked to one another;
(b) said cell penetrating peptide domain is adapted to penetrate a mammalian cell membrane, and thereby to cause the moieties to which said cell penetrating domain is covalently linked to be internalized by a mammalian cell;
(c) said kinase domain has 95% or greater sequence identity with a naturally-occurring mammalian Tousled-like kinase 1 kinase domain with or a naturally-occurring mammalian Tousled-like kinase 2 kinase domain, and said kinase domain is adapted to repair genotoxic damage to a mammalian cell's DNA;
(d) each of said at least one proteolytic cleavage sites is adapted to be proteolytically cleaved by at least one naturally-occurring mammalian matrix metalloproteinase, or by naturally-occurring mammalian furin, or both; and
(e) either condition (i) or condition (ii) is true:
(i) said proteolytic cleavage site is located between said cell penetrating peptide domain and said kinase domain; whereby cleavage of said proteolytic cleavage site causes said cell penetrating peptide domain and said kinase domain no longer to be covalently linked to one another; or
(ii) said proteolytic cleavage site is located within said kinase domain, such that when intact the proteolytic cleavage site does not substantially inhibit the kinase activity of said kinase domain; and such that proteolysis of the PRELIMINARY AMENDMENT Page 3 of 6 proteolytic cleavage site substantially reduces the kinase activity of said kinase domain.

2. The protein of claim 1, wherein said cell penetrating peptide domain is selected from the group consisting of the TAT peptide residues 47-57 (SEQ ID NO: 80); or SEQ ID NO: 77, or another polyarginine; or a modification of the TAT peptide residues 47-57 (SEQ ID NO: 80), in which modified peptide one or more non-cationic amino acid residues have been replaced with a cationic amino acid.

3. The protein of claim 1, wherein said kinase domain is selected from the group consisting of Tousled-like kinase 1, Tousled-like kinase 1B, Tousled-like kinase 2, or another serine-threonine kinase.

4. The protein of claim 1, wherein said proteolytic cleavage site is sensitive to cleavage both by MMP1 and by MMP2.

5. The protein of claim 1, wherein said proteolytic cleavage site is sensitive to cleavage by furin.

6. The protein of claim 1, wherein the sequence of said protein is SEQ ID NO: 3.

7. The protein of claim 1, wherein the sequence of said protein is SEQ ID NO: 4.

8. A method for treating a cancerous tumor in a mammal and inhibiting the growth of the tumor, while simultaneously protecting non-cancerous cells in the vicinity of the tumor from adverse effects of a cancer therapy; wherein the cells of the tumor overexpress a matrix metalloproteinase, or the cells of the tumor overexpress furin, or both; wherein said method comprises co-administering to the tumor a cancer therapy and the recombinant protein of claim 1; wherein the cancer therapy comprises one more of ionizing radiation, targeted particle therapy, radionuclide treatment, chemotherapy, or photodynamic therapy; wherein the overexpressed matrix metalloproteinase or the overexpressed furin differentially causes cleavage of the proteolytic cleavage site on tumor cells, inside tumor cells, or in the immediate environment of tumor cells to occur at a substantially higher rate than cleavage of the proteolytic cleavage site on non-cancerous cells, inside non-cancerous cells, or in the immediate environment of non-cancerous cells; whereby a substantially higher fraction of the recombinant protein is cleaved on, inside, or in the immediate environment of tumor cells than is cleaved on, inside, or in the immediate environment of non-cancerous cells; whereby the uncleaved recombinant protein differentially provides greater protection to non-cancerous cells from the effects of the therapy than any protection provided to tumor cells, or the cleaved recombinant protein differentially imparts greater sensitization to tumor cells to the therapy than any sensitization imparted to non-cancerous cells, or both.

9. The method of claim 8, wherein said cell penetrating peptide domain is selected from the group consisting of the TAT peptide residues 47-57 (SEQ ID NO: 80); or SEQ ID NO: 77, or another polyarginine; or a modification of the TAT peptide residues 47-57 (SEQ ID NO: 80), in which modified peptide one or more non-cationic amino acid residues have been replaced with a cationic amino acid.

10. The method of claim 8, wherein the kinase domain is selected from the group consisting of Tousled-like kinase 1, Tousled-like kinase 1B, Tousled-like kinase 2, or another serine-threonine kinase.

11. The method of claim 8, wherein the proteolytic cleavage site is sensitive to cleavage both by MMP1 and by MMP2.

12. The method of claim 8, wherein the proteolytic cleavage site is sensitive to cleavage by furin.

13. The method of claim 8, wherein the sequence of the protein is SEQ ID NO: 3.

14. The method of claim 8, wherein the sequence of the protein is SEQ ID NO: 4.

15. The protein of claim 1, wherein the sequence of said protein is SEQ ID NO: 89.

16. The method of claim 8, wherein the sequence of the protein is SEQ ID NO: 89.
